SEQUENCE LISTING (8 sequences)

SEQ ID NO: 1 - protein, 204 aa, Homo sapiens
GGSGFPGFGVGVGGIPGVAGVPGVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPAIGPEAQAAAAAKAAKYGVGTPAAAAAKAAAKAAQFGLVPGVGVAPGVGVAPGVGVAPGVGLAPGVGVAPGVGVAPGVGVAPAIGP

SEQ ID NO: 2 - protein, 162 aa, Ovis aries
GSQQPGVPLGYPIKAPKLPGGYGLPYSTGKLPYGYGPGGVAGAAGKAGYPTGTGVGPQAAAAAAKAAAKLGAGGAGVLPGVGVGGAGIPGAPGAIPGIGGIAGAGAPDAAAAAAAAAKAAKYGGAGVFGPGVVGVPGAGVPGVGVPGVGVPGVGVPGVGVPG

SEQ ID NO: 3 - protein, 96 aa, Homo sapiens
GSGQPGVPLGYPIKAPKLPGGYGLPYGVGPAAAAAAAKYAAKYAQFGLGGQPGVPLGYPIKAPKLPGGYGLPYGVGPAAAAAAAKAAAKAAQFGLG

SEQ ID NO: 4 - protein, 96 aa, Homo sapiens; identical to SEQ ID NO: 3

SEQ ID NO: 5 - protein, 96 aa, Homo sapiens
GSGQPGVPLGYPIKAPKLPGGYGLPYGVGPAAAAAAAKAAAKAAQFGLGGQPGVPLGYPIKAPKLPGGYGLPYGVGPAAAAAAAKYAAKYAQFGLG

SEQ ID NO: 6 - protein, 96 aa, Homo sapiens
GSGQPGVPLGYPIKAPKLPGGYGLPYGVGPAAAAAAAKYAAKYAQFGLGGQPGVPLGYPIKAPKLPGGYGLPYGVGPAAAAAAAKYAAKYAQFGLG

SEQ ID NO: 7 - protein, 10 aa, Homo sapiens
PGAGVKPGKV

SEQ ID NO: 8 - protein, 9 aa, Homo sapiens
AAAAAKAAK

(19) United States
(12) Patent Application Publication
(10) Pub. No.: US 2022/0387665 A1
(43) Pub. Date: Dec. 8, 2022

(54) METHODS AND COMPOSITIONS FOR MENISCAL REPAIR USING BIOADHESIVE PROTEINS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Ningning Mussomeli, Round Rock, TX (US); Stephen Santangelo, Sturbridge, MA (US); Hans Hoppe, Cambridgeshire (GB)

(73) Assignee: Smith & Nephew, Inc., Memphis, TN (US)

(21) Appl. No.: 17/826,623

(22) Filed: May 27, 2022

Related U.S. Application Data
(60) Provisional application No. 63/196,344, filed on Jun. 3, 2021.

(51) Int. Cl. A61L 24/10; A61L 24/04; A61L 27/36; C07K 14/435; C09J 189/00

(52) U.S. Cl. CPC A61L 24/108; A61L 24/043; A61L 27/3633; C07K 14/43509; C09J 189/00; A61L 2430/06

(57) ABSTRACT

Compositions and methods are directed to engineered extracellular matrix protein—mussel foot protein fusions for use as a bioadhesive for repairing tissues. The compositions have one or more of: (i) at least one hydrophobic region; (ii) at least one crosslinking region; (iii) at least one tyrosine residue accessible to be enzymatically modified to a DOPA or TOPA side chain; (iv) at least one mussel foot protein; (v) at least one mussel foot protein loop; (vi) at least one human extracellular protein loop; or (vii) at least one of the following sequences: SEQ ID NO: 1, SEQ ID NO: 2, SEQ ID NO: 3, SEQ ID NO: 4, SEQ ID NO: 5, or SEQ ID NO: 6. The elastin-like polypeptide includes at least one non-naturally occurring amino acid or sequence alteration.

### CROSS-REFERENCE TO RELATED APPLICATIONS

This application claims priority to and benefit of U.S. Provisional Application No. 63/196,344, filed Jun. 3, 2021, entitled METHODS AND COMPOSITIONS FOR MENISCAL REPAIR USING BIOADHESIVE PROTEINS, the entire contents of which are incorporated herein by reference for all purposes.

### BACKGROUND

There has been a wealth of recent interest in the development of adhesive materials that function in wet or underwater environments. In particular, much of this focus has been placed on development of adhesives for biomedical applications. Suitable biomedical adhesives would have an immense impact on public health and the economy. Each year, over 230 million major surgeries are performed worldwide, and over 12 million traumatic wounds are treated in the U.S. alone. Approximately 60% of these wounds are closed using mechanical methods such as sutures and staples. Sutures and staples have several disadvantages relative to adhesives, including patient discomfort, higher risk of infection, and the inherent damage to surrounding healthy tissue.

Current FDA-approved adhesives and sealants face several challenges. First, numerous adhesives exhibit toxic characteristics. For example, cyanoacrylate-based adhesives like Dermabond® and SurgiSeal® can only be applied topically due to carcinogenic degradation products. Fibrin sealants like Tisseel and Artiss are derived from blood sources and therefore carry the potential for blood-borne pathogen transmission. Poly(ethylene glycol) (PEG) adhesives are approved as suture sealants but, due to intense swelling when wet, have the potential to cause inflammatory responses. TissuGlu®, which uses subcutaneous implantation, induced seroma formation in 22% of patients. More importantly, most of these adhesives do not possess strong adhesion in wet environments and are not approved for application in wound closure.

In approaching the challenge of developing a strong adhesive for wet applications, many researchers have been inspired by natural glues. Specifically, materials derived from organisms such as sandcastle worms and mussels have demonstrated underwater application and bonding. Both sandcastle worms and mussels produce proteins containing the non-canonical amino acid 3,4-dihydroxyphenylalanine (DOPA), which has been shown to provide adhesion strength, even in wet environments.

Biomaterials as a fast, efficient way to repair wounded tissue in sports medicine applications, such as meniscal repair, and support tissue healing are gaining attention. However, the combination of features required for optimal wound healing support—wet adhesion, biocompatibility, appropriate resorption profile, high strength, fast cure time and functions to sealant against the aqueous environment in joint space—are not currently available in either one material or in material combinations.

Currently over 85% of meniscal tears are treated with resection or left in situ instead of repair due to poor healing ability, especially for tears in red-white or white-white zones. Meniscectomy significantly increases the likelihood of osteoarthritis post-surgery.

The current standard of care for meniscal tear repair is mechanical fixation methods such as anchors and sutures. There has been some development in the research of wet adhesives in nature, such as mussel adhesion proteins (MAP), as well as other biomimetic adhesives in the past few decades. However, these materials either likely cause significant immune response in humans, or do not fully utilize the potential of MAP.

### SUMMARY

A class of recombinant proteins as bioadhesives that realizes all features required for optimal wound healing support—that is, wet adhesion, biocompatibility, appropriate resorption profile, high strength, fast cure time, and sealant against the aqueous environment in joint space—is disclosed. These proteins are designed based on polypeptides from human and/or sheep elastins and are functionalized by enzymatically or chemically converting the tyrosine residues to 3,4-dihydroxyphenylalanine (DOPA) groups or 3,4,5-trihydroxyphenylalanine (TOPA) groups to achieve adhesiveness similar to MAP. Other proteins disclosed herein are fusion proteins designed based on polypeptides from human and/or sheep elastins combined with polypeptides from mussel foot proteins or human extracellular protein loops that resemble mussel foot protein. By combining the mechanisms of fibril formation of elastin with the ability of the DOPA groups to form adhesion to various wet, organic or inorganic materials, these materials form tough, robust adhesives that minimize immunogenicity.

In embodiments, a composition of an elastin-like polypeptide of this disclosures includes one or more of: (i) at least one hydrophobic region; (ii) at least one crosslinking region; (iii) at least one tyrosine residue accessible to be enzymatically modified to a DOPA or TOPA side chain; (iv) at least one mussel foot protein; (v) at least one mussel foot protein loop; (vi) at least one human extracellular protein loop; or (vii) at least one of the following sequences: SEQ ID NO: 1, SEQ ID NO: 2, SEQ ID NO: 3, SEQ ID NO: 4, SEQ ID NO: 5, or SEQ ID NO: 6. The elastin-like polypeptide includes at least one non-naturally occurring amino acid or sequence alteration. In embodiments, the DOPA or TOPA modification occurs by co-expressing the peptide with a tyrosinase in E. coli. In embodiments, the DOPA or TOPA modification occurs by adding the tyrosinase enzyme to a solution containing the elastin-like polypeptide. In embodiments, the elastin-like polypeptide is part of a DOPA or TOPA functionalized hydrogel, optionally comprising other synthetic polymers. In embodiments, the elastin-like polypeptide contains binding sites to cytokines or cell surface receptors.

In further embodiments, a method of bonding tissues of this disclosure includes the step of applying an elastin-like polypeptide as a bioadhesive to at least one tissue in a wet environment. The elastin-like polypeptide includes one or more of (i) at least one hydrophobic region; (ii) at least one crosslinking region; (iii) at least one tyrosine residue accessible to be enzymatically modified to a DOPA side chain; (iv) at least one mussel foot protein; (v) at least one mussel foot protein loop; (vi) at least one human extracellular protein loop; or (vii) at least one of the following sequences: SEQ ID NO: 1, SEQ ID NO: 2, SEQ ID NO: 3, SEQ ID NO: 4, SEQ ID NO: 5, or SEQ ID NO: 6. The elastin-like polypeptide includes at least one non-naturally occurring amino acid or sequence alteration. In embodiments, the DOPA or TOPA modification occurs by co-expressing the peptide with a tyrosinase in E. coli. In embodiments, the DOPA or TOPA modification occurs by adding the tyrosinase enzyme to a solution containing the elastin-like polypeptide. In embodiments, the elastin-like polypeptide is part of a DOPA or TOPA functionalized hydrogel, optionally comprising other synthetic polymers. In embodiments, the elastin-like polypeptide contains binding sites to cytokines or cell surface receptors. In embodiments, the wet environment comprises a solution, environmental humidity, or body fluids. In embodiments, the method treats a meniscal tear. In embodiments, the elastin-like polypeptide is delivered to the site of the tear arthroscopically and forms a layer that covers and seals the tear from the synovial fluid. In embodiments, the method treats a wound. In embodiments, the wound occurs due to surgery, orthopedic stress, or sports.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1 illustrates a representative application of the bioadhesives in meniscal repair as described in the present disclosure;

FIGS. 2A-2F illustrate various construct designs of the elastin-like Mussel Foot Protein inspired bioadhesive proteins; and

FIGS. 3A-3F illustrate specific embodiments of elastin-like bioadhesive proteins as described in the present disclosure.

### DETAILED DESCRIPTION

Embodiments of the present disclosure relate to methods and compositions for meniscal repair using bioadhesive protein. The design of such a protein can vary. Aside from meniscal repair, the bioadhesive may have broad applications in, but not limited to, sports medicine, orthopedics, wound care, and ENT surgeries. By adjusting the design of the protein, as well as titrating multiple proteins with various structures, properties of the adhesives such as the toughness, adhesion strength, and permeability can be tailored to specific applications. Further, the molecules may be modified to include binding sites to certain cytokines, including but not limited to IL-1 and TNF-α, to mediate inflammatory response and aid the healing process.

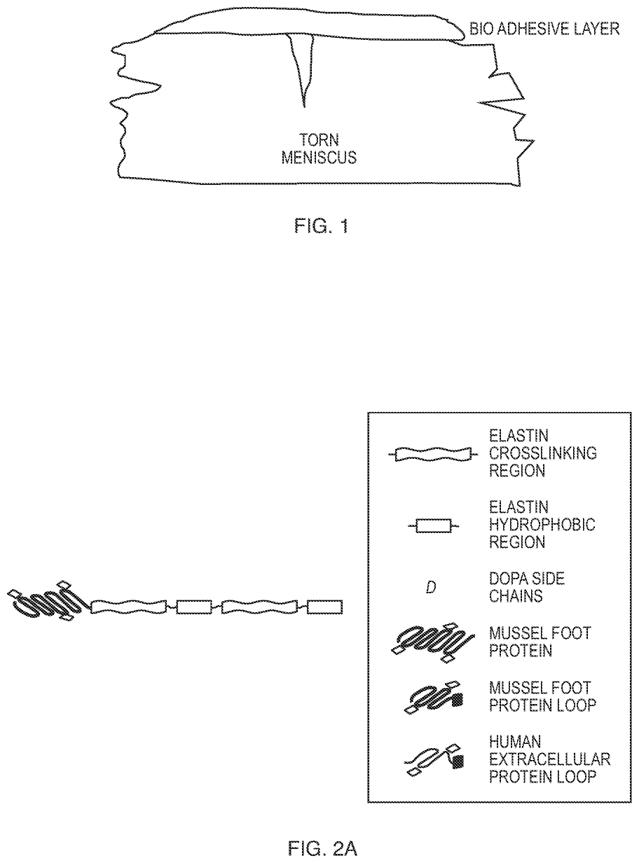

A representative scenario of the application of this adhesive in meniscal repair is shown in FIG. 1. The adhesive is delivered to the site of the tear arthroscopically and forms a layer that covers and seals the tear from the synovial fluid. The layer provides mechanical strength and toughness to mechanically fix the tear, with or without traditional fixation methods such as sutures and anchors. The adhesive layer protects any blood clot that might have formed inside the tear, and blocks out or binds to some of the cytokine molecules such as IL-1 that are over produced in the synovial fluid after injury. By physically protecting the wound site and altering the local biochemical environment, the adhesive provides a more favorable environment for the tissue to heal. In addition, the adhesive can serve as a vehicle to deliver or be used together with active biologics, such as growth factors, to aid healing.

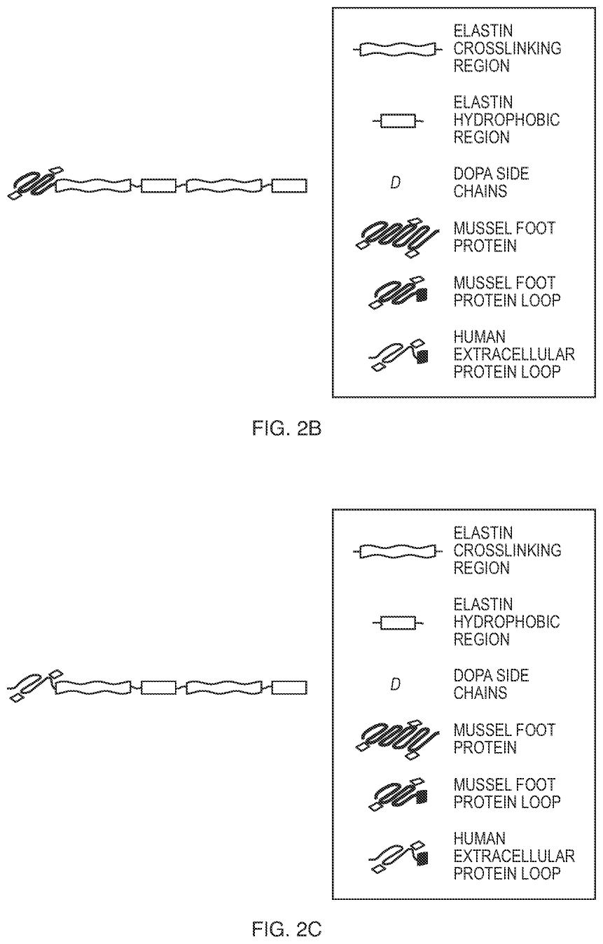

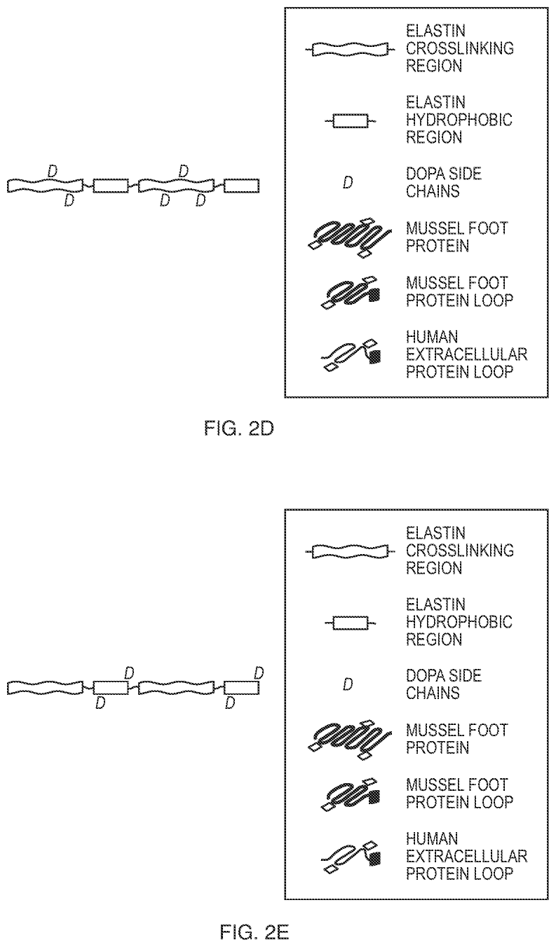

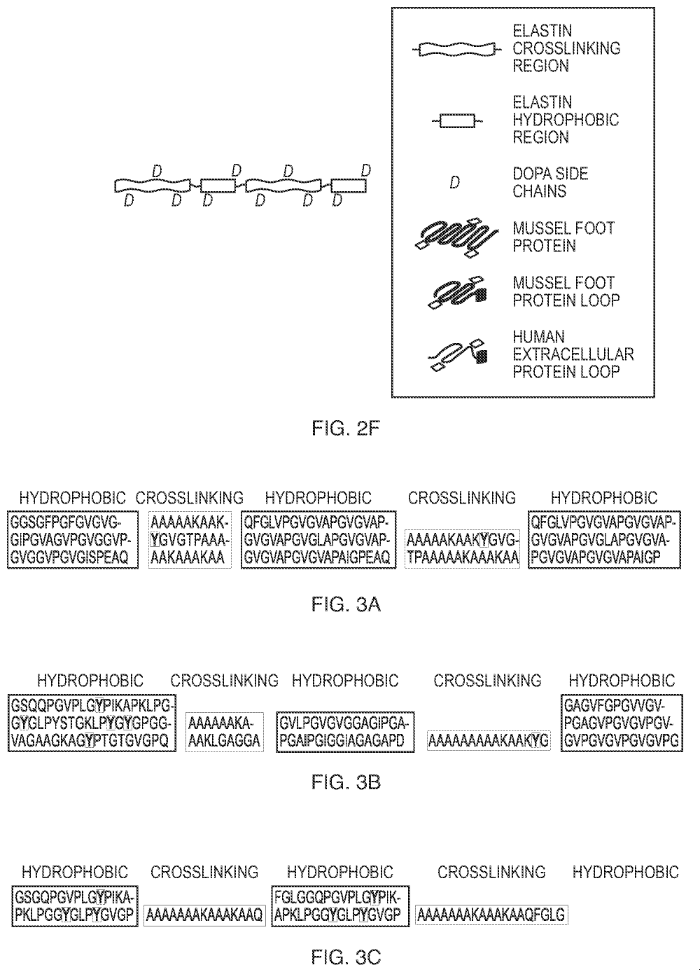

FIG. 2A shows a fusion protein constructed with elastin-like peptides and a Mussel Foot Protein enriched in Y, K, S and P amino acids. FIG. 2B shows a fusion protein constructed with elastin-like peptides and a mussel foot protein loop enriched in Y, K, S and P amino acids. FIG. 2C shows a fusion protein construct with elastin-like peptides and a human extracellular protein loop enriched in Y, K, S, and P amino acids sharing sequence similarity with the mussel foot protein loops of FIG. 2B. FIG. 2D shows an elastin construct with the crosslinking regions containing modified 3,4-dihydroxyphenylalanine (DOPA) side chains. FIG. 2E shows an elastin construct with the hydrophobic regions containing modified DOPA side chains. FIG. 2F shows an elastin construct with both the crosslinking regions and the hydrophobic regions containing modified DOPA side chains.

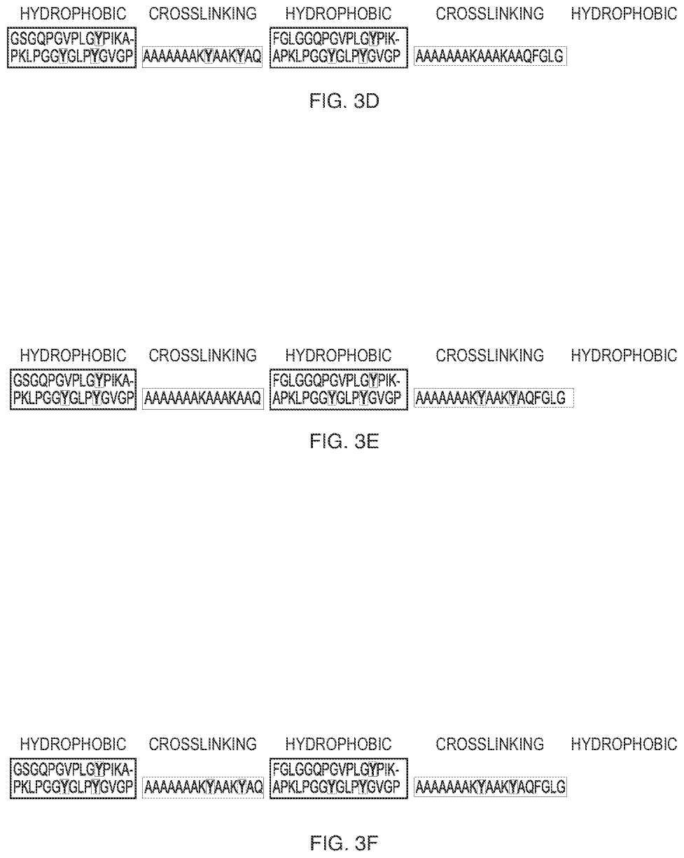

FIG. 3A (SEQ ID NO: 1) shows elastin-like bioadhesive proteins based on human elastin sequences. FIG. 3B (SEQ ID NO: 2) shows elastin-like bioadhesive proteins based on sheep elastin sequences. FIG. 3C (SEQ ID NO: 3) shows elastin-like bioadhesive proteins based on common sequences found in human and sheep elastins. FIG. 3D (SEQ ID NO: 4) shows elastin-like bioadhesive proteins based on common sequences found in human and sheep elastins but with two Alanine residues in the first crosslinking region replaced with Tyrosine residues. FIG. 3E (SEQ ID NO: 5) shows elastin-like bioadhesive proteins based on common sequences found in human and sheep elastins but with two Alanine residues in the second crosslinking region replaced with Tyrosine residues. FIG. 3F (SEQ ID NO: 6) shows elastin-like bioadhesive proteins based on common sequences found in human and sheep elastins but with two Alanine residues in both crosslinking regions replaced with Tyrosine residues.

In one embodiment, a composition of an elastin-like polypeptide comprises: (i) at least one hydrophobic region; (ii) at least one KP-type cross-linking region (e.g., SEQ ID NO: 7 PGAGVKPGKV) or KA-type cross-linking region (e.g., SEQ ID NO: 8 AAAAAKAAK) and/or (iii) at least one tyrosine residue accessible to be enzymatically modified to a DOPA or TOPA side chain. In other embodiments, a composition of an elastin-like polypeptide further comprises (iv) at least one mussel foot protein; (v) at least one mussel foot protein loop; and/or (vi) at least one human extracellular protein loop. In yet other embodiments, a composition of an elastin-like polypeptide comprises at least one of the following sequences: SEQ ID NO: 1, SEQ ID NO: 2, SEQ ID NO: 3, SEQ ID NO: 4, SEQ ID NO: 5, or SEQ ID NO: 6.

In one embodiment of a composition, DOPA or TOPA modification occurs by co-expressing the peptide with a tyrosinase in E. coli.

In one embodiment of a composition, DOPA or TOPA modification occurs by adding the tyrosinase enzyme to a solution containing the elastin-like polypeptide.

In one embodiment of a composition, the elastin-like polypeptide is part of a DOPA or TOPA functionalized hydrogel.

In one embodiment of a composition, the elastin-like polypeptide contains binding sites to cytokines or cell surface receptors.

In one embodiment, a method of applying an elastin-like polypeptide comprises: (i) at least one hydrophobic region; (ii) at least one crosslinking region; and/or (iii) at least one tyrosine residue accessible to be enzymatically modified to a DOPA or TOPA side chain. In other embodiments, a composition of an elastin-like polypeptide further comprises (iv) at least one mussel foot protein; (v) at least one mussel foot protein loop; and/or (vi) at least one human extracellular protein loop. In yet other embodiments, a composition of an elastin-like polypeptide comprises at least one of the following sequences: SEQ ID NO: 1, SEQ ID NO: 2, SEQ ID NO: 3, SEQ ID NO: 4, SEQ ID NO: 5, or SEQ ID NO: 6.

In one embodiment of a method, DOPA modification occurs by co-expressing the peptide with a tyrosinase in E. coli.

In one embodiment of a method, DOPA modification occurs by adding the tyrosinase enzyme to a solution containing the elastin-like polypeptide.

In one embodiment of a method, the elastin-like polypeptide is part of a DOPA functionalized hydrogel, optionally comprising other synthetic polymers.

In one embodiment of a method, the elastin-like polypeptide contains binding sites to cytokines or cell surface receptors.

In one embodiment of a method, the wet environment comprises a solution, environmental humidity, or wet tissue.

In one embodiment, the method treats a meniscal tear.

In one embodiment, the method treats a wound.

In one embodiment, the wound occurs due to surgery, orthopedic stress, or sports.

In the instant disclosure, an “elastin-like” polypeptide or protein is a sequence of amino acids with either the exact sequence of a portion of a human elastin, or similar enough to a human elastin sequence so as to not induce immunogenicity, e.g. a sheep elastin or a portion thereof.

In the instant disclosure, an “elastin-like polypeptide including at least one non-naturally occurring amino acid or sequence alteration” is a sequence of amino acids that does not occur in nature. Embodiments of sequence alterations include amino acid deletions, additions and substitutions. Embodiments of non-naturally occurring amino acid include amino acid substitutions and chemical or enzymatic changes to naturally occurring amino acids (i.e., those that occur in the native sequence) or non-naturally occurring amino acids (e.g. substitutions).

In the instant disclosure, the polypeptides are constructed with alternating hydrophobic loops and alpha helix crosslinking regions to facilitate elastin fibril formations.

In the instant disclosure, the polypeptides include a high number of tyrosine residues, either within the hydrophobic regions, the crosslinking regions, in an additional sequence region or a combination of these regions, which are accessible chemically to be enzymatically or chemically modified with DOPA or TOPA side chains. The additional sequence regions can include mussel foot proteins or peptides, mussel foot protein loops and/or human extracellular protein loops.

In the instant disclosure, a “bioadhesive” is a biological glue for use in a biomedical application. The present disclosure provides elastin-like proteins having adhesive properties better than other biological glues known in the art. The elastin-like proteins of the present disclosure are advantageous for use in wet adhesion. Additionally, the elastin-like proteins of the present disclosure show high cytocompatability and are appropriate for use in biomedical applications. Moreover, the elastin-like protein/mussel adhesive protein (MAP) fusions (elastin-MAP fusion proteins) exhibit high strength as bioadhesives due to the elastin-like matrix and the strong adhesiveness to wet surfaces of the MAP portions. In addition, DOPA- or TOPA-mediated cross-links of MAP-like peptides to human tissue proteins are expected to escape direct immune surveillance to a greater degree than non-crosslinked peptides of the same sequence.

Current adhesives in the art need to be applied to a dry or almost dry surface in order to have the adhesive strength required for many applications, particularly those in the biomedical area. For example, it is desirable to use adhesives in place of staples or sutures. However, the adhesives currently being used are limited to applications where the surfaces to be bonded can be dried. In contrast, the elastin-like polypeptide adhesives of the present disclosure may be applied underwater, in high humidity and on wet tissue and still form a strong adhesive bond.

In the instant disclosure, “DOPA” refers to 3,4-dihydroxyphenylalanine formed by hydroxylation of tyrosine and “TOPA” refers to 3,4,5-trihydroxyphenylalanine formed by hydroxylation of tyrosine. Methods of DOPA and TOPA modification include, but are not limited to: co-expressing the tyrosine containing peptide with a tyrosinase in E. coli, adding a tyrosinase enzyme to a solution containing the elastin-like polypeptide or chemical modification methods.

While various embodiments of protein-based adhesives and methods of producing the same have been described in considerable detail herein, the embodiments are merely offered as non-limiting embodiments of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

The terms comprise, include, and/or plural forms of each are open ended and include the listed parts and can include additional parts that are not listed. The term and/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

1. A composition of an elastin-like polypeptide comprising one or more of:
(i) at least one hydrophobic region;
(ii) at least one crosslinking region;
(iii) at least one tyrosine residue accessible to be enzymatically modified to a DOPA or TOPA side chain;
(iv) at least one mussel foot protein;
(v) at least one mussel foot protein loop;
(vi) at least one human extracellular protein loop; or
(vii) at least one of the following sequences: SEQ ID NO: 1, SEQ ID NO: 2, SEQ ID NO: 3, SEQ ID NO: 4, SEQ ID NO: 5, or SEQ ID NO: 6;
wherein the elastin-like polypeptide includes at least one non-naturally occurring amino acid or sequence alteration.

2. The composition of claim 1, wherein the DOPA or TOPA modification occurs by co-expressing the peptide with a tyrosinase in E. coli.

3. The composition of claim 1, wherein the DOPA or TOPA modification occurs by adding the tyrosinase enzyme to a solution containing the elastin-like polypeptide.

4. The composition of claim 1, wherein the elastin-like polypeptide is part of a DOPA or TOPA functionalized hydrogel, optionally comprising other synthetic polymers.

5. The composition of claim 1, wherein the elastin-like polypeptide contains binding sites to cytokines or cell surface receptors.

6. A method of bonding tissues comprising the step of:
applying an elastin-like polypeptide as a bioadhesive to at least one tissue in a wet environment, said elastin-like polypeptide comprising one or more of:
(i) at least one hydrophobic region;
(ii) at least one crosslinking region;
(iii) at least one tyrosine residue accessible to be enzymatically modified to a DOPA side chain;
(iv) at least one mussel foot protein;
(v) at least one mussel foot protein loop;
(vi) at least one human extracellular protein loop; or
(vii) at least one of the following sequences: SEQ ID NO: 1, SEQ ID NO: 2, SEQ ID NO: 3, SEQ ID NO: 4, SEQ ID NO: 5, or SEQ ID NO: 6;
wherein the elastin-like polypeptide includes at least one non-naturally occurring amino acid or sequence alteration.

7. The method of claim 6, wherein the DOPA or TOPA modification occurs by co-expressing the peptide with a tyrosinase in E. coli.

8. The method of claim 6, wherein the DOPA or TOPA modification occurs by adding the tyrosinase enzyme to a solution containing the elastin-like polypeptide.

9. The method of claim 6, wherein the elastin-like polypeptide is part of a DOPA or TOPA functionalized hydrogel, optionally comprising other synthetic polymers.

10. The method of claim 6, wherein the elastin-like polypeptide contains binding sites to cytokines or cell surface receptors.

11. The method of claim 6, wherein the wet environment comprises a solution, environmental humidity, or body fluids.

12. The method of claim 11, wherein the method treats a meniscal tear.

13. The method of claim 12, wherein the elastin-like polypeptide is delivered to the site of the tear arthroscopically and forms a layer that covers and seals the tear from the synovial fluid.

14. The method of claim 11, wherein the method treats a wound.

15. The method of claim 13, wherein the wound occurs due to surgery, orthopedic stress, or sports.
